(19) United States
(12) Patent Application Publication
(10) Pub. No.: US 2021/0023387 A1
(43) Pub. Date: Jan. 28, 2021

(54) Electronic Pad for Light Treatment

(71) Applicant: Matthew C. DeBOW, Buda, TX (US)

(72) Inventor: Matthew C. DeBOW, Buda, TX (US)

(21) Appl. No.: 16/067,552

(22) PCT Filed: Jan. 17, 2017
(86) PCT No.: PCT/US2017/013809

§ 371 Date: Jun. 29, 2018

Related U.S. Application Data
(60) Provisional application No. 62/280,118, filed on Jan. 18, 2016.

(51) Int. Cl. A61N 5/06

(52) U.S. Cl. CPC A61N 5/0613; A61N 2005/0661; A61N 2005/0663; A61N 2005/0647; A61N 2005/0632; A61N 2005/0652; A61N 2005/066

(57) ABSTRACT

A pad having a circuit board and multiple light sources to emit light having a wavelength in a range of 300 nanometers (nm) to 1100 nm for light therapy treatment of a human. A front exterior layer may have multiple raised portions to cover the multiple light sources, respectively, the front exterior layer to face a body part of the human in treatment. The pad may be conformable to the body part.

### CROSS-REFERENCE TO RELATED APPLICATION

This application claims the benefit of U.S. Provisional Patent Application No. 62/280,118, filed on Jan. 18, 2016, the contents of which are incorporated by reference herein in their entirety herein.

### TECHNICAL FIELD

The present techniques relate generally to an electronic pad for light treatment, and more particularly but not limited to a flexible electronic pad having a flexible circuit and multiple light sources to apply light to a treatment subject.

### BACKGROUND ART

Pain is as old as mankind Attempts to mitigate pain led to a predominately pharmaceutical response. Initially, opiate drugs were prescribed for pain management, but had addictive qualities. Currently, most households possess one or more containers of aspirin or ibuprofen, which are a typical choice in the home for most pain issues. As can be expected, there is an ongoing need to address and improve implementations for reducing pain and promoting healing.

### SUMMARY

An aspect relates to a pad including: an internal layer comprising a circuit board and having multiple light sources to emit light having a wavelength in a range of 300 nanometers (nm) to 1100 nm for light therapy treatment of a human; a power input jack coupled to the circuit board to receive power to the pad, a front exterior layer to face a body part of the human in treatment; and a back exterior layer. The back exterior layer may coupled to the front exterior layer and the two exterior layers in combination at least partially encase the internal layer. The pad may be facemask pad or a headgear pad. The pad may be shaped to fit a specific body part.

Another aspect relates to method of operating a pad in light therapy, including: positioning a surface of a front exterior layer of the pad against a body part of a human, coupling the pad to a power supply; and turning on the pad to emit light from a plurality of light sources on internal circuitry of the flexible pad, the light having a wavelength in a range of 300 nanometers (nm) to 1000 nm and directed through the front exterior layer to the body part.

Yet another aspect relates to a method of manufacturing a pad for light therapy of a human, the method including: obtaining a base polymeric material; obtaining a circuit board comprising at least 3 light sources to emit light at a wavelength in a range of 300 nanometers (nm) to 1100 nm; and forming the base polymeric material around the circuit board. The forming may include: forming a front exterior layer have raised portions to accommodate the light sources, wherein the light sources to emit the light through the front exterior layer to a body portion of the human for the light therapy; and forming a back exterior layer.

### BRIEF DESCRIPTION OF DRAWINGS

Advantages of the invention may become apparent upon reading the following detailed description and upon reference to the drawings in which:

FIGS. 1A-1F are diagrams of pads for light treatment in accordance with embodiments of the present techniques;

FIG. 1G is a side cross-sectional view of a pad for light treatment in accordance with embodiments of the present techniques;

FIG. 1 is perspective views of a pad for light treatment in accordance with embodiments of the present techniques;

FIG. 2 is an exploded view of the pad of FIG. 1 in accordance with embodiments of the present techniques;

FIG. 3 is a diagram of exemplary pad with various power-supply options in accordance with embodiments of the present techniques;

FIG. 4 is a block flow diagram of method of operating a pad for light therapy in accordance with embodiments of the present techniques;

FIG. 5 is a block flow diagram of method of manufacturing a pad for light therapy in accordance with embodiments of the present techniques;

FIG. 6 is a perspective view of a pad in accordance with embodiments of the present techniques;

FIG. 7 is a diagrammatical representation of a pad applied to the back of a person in accordance with embodiments of the present techniques;

FIG. 8A is a diagrammatical representation of a pad applied to a knee of a person in accordance with embodiments of the present techniques;

FIG. 8B is a diagrammatical representation of a pad applied to a shoulder of a person in accordance with embodiments of the present techniques;

FIG. 9A is a diagram of a human subject and example body parts or portions that may be treated with a pad in accordance with embodiments of the present techniques;

FIG. 9B is a diagram of a control unit in accordance with embodiments of the present techniques;

FIG. 10 is a perspective view of a pad in accordance with embodiments of the present techniques;

FIGS. 11A and 11B are diagrammatical views of a facemask pad in accordance with embodiments of the present techniques;

FIG. 12 is a perspective view of the facemask pad of FIG. 11 being applied to a face of a human in accordance with embodiments of the present techniques;

FIGS. 13A and 13B are diagrammatical representations of belts in accordance with embodiments of the present techniques; and

FIGS. 14-20 are perspective views of several different flexible pads to fit different body parts of a human, respectively, in accordance with embodiments of the present techniques.

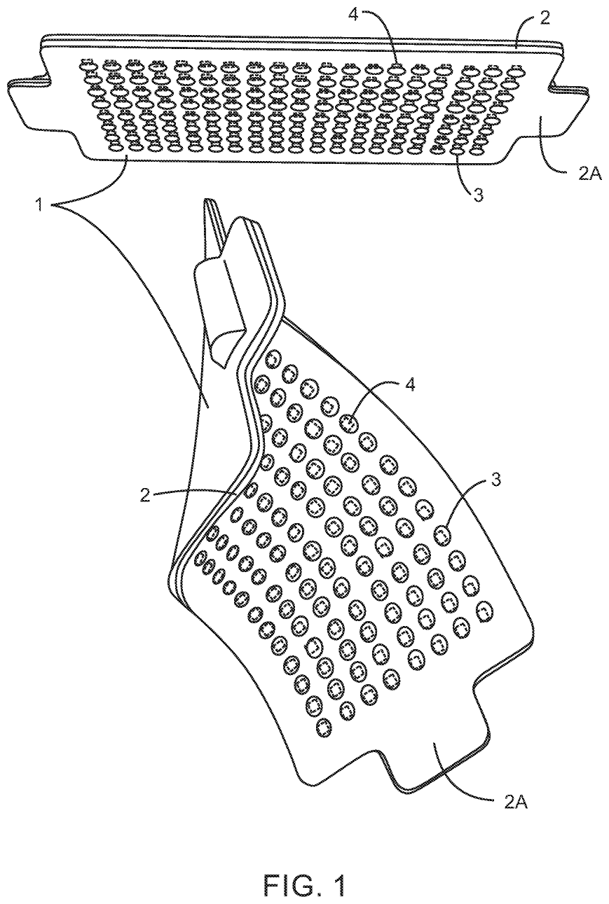

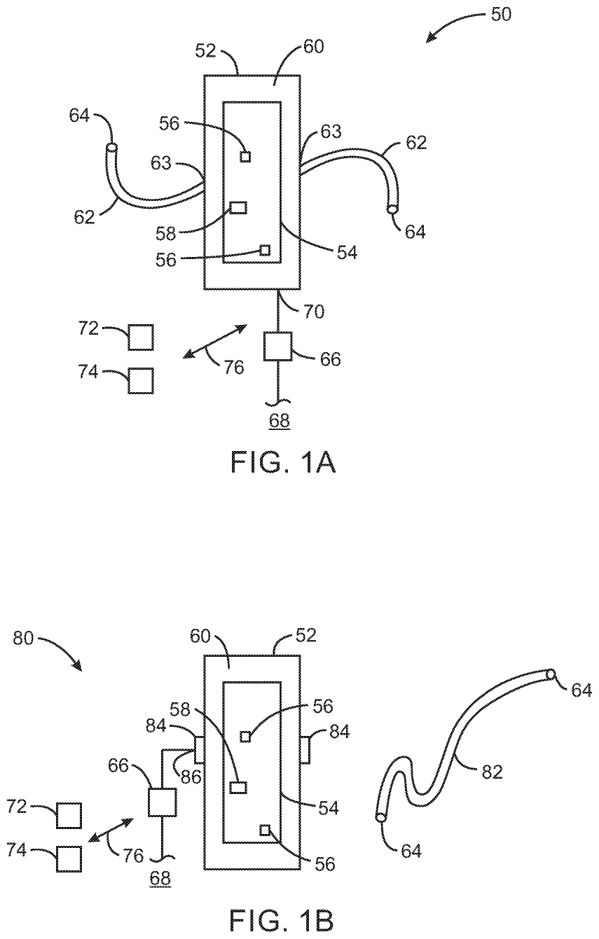

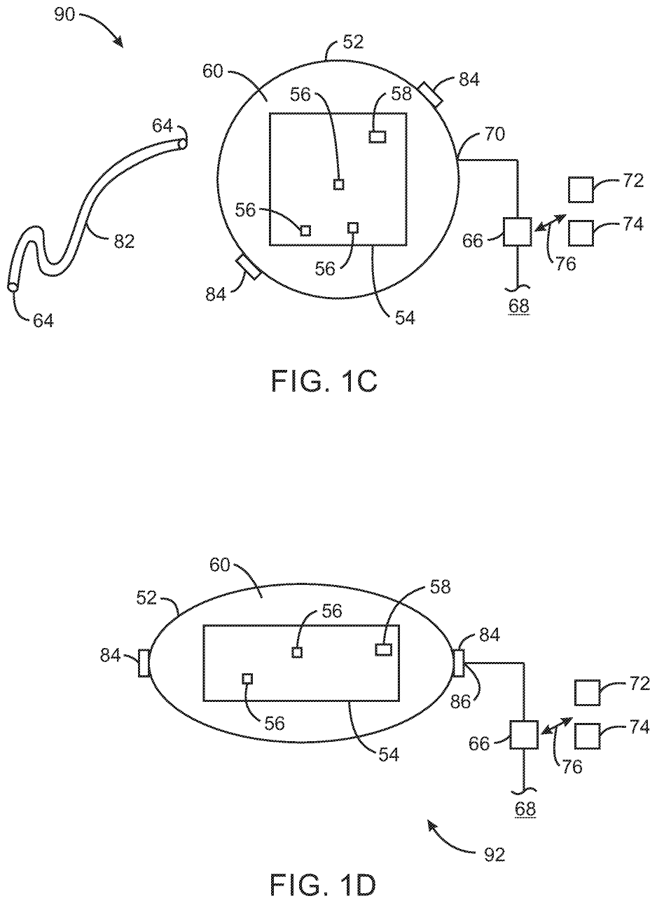

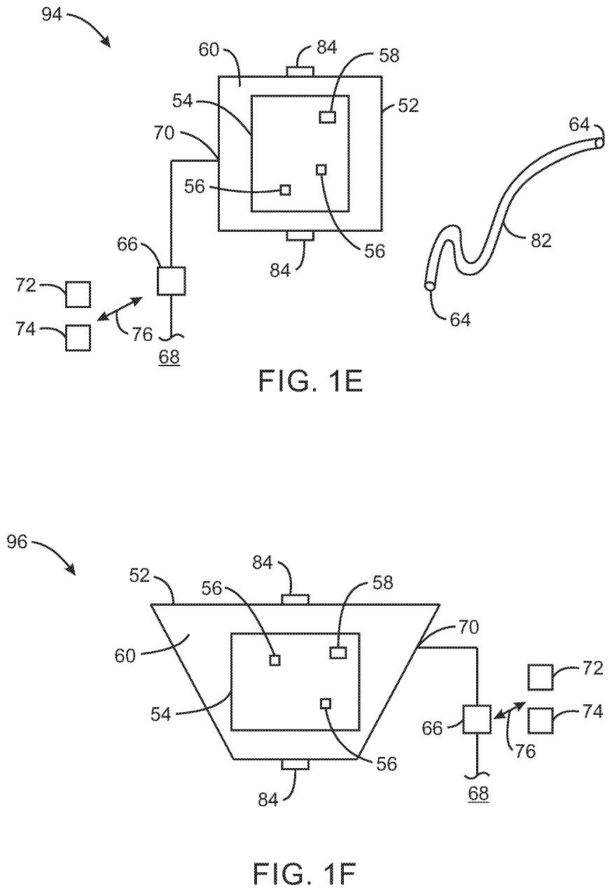

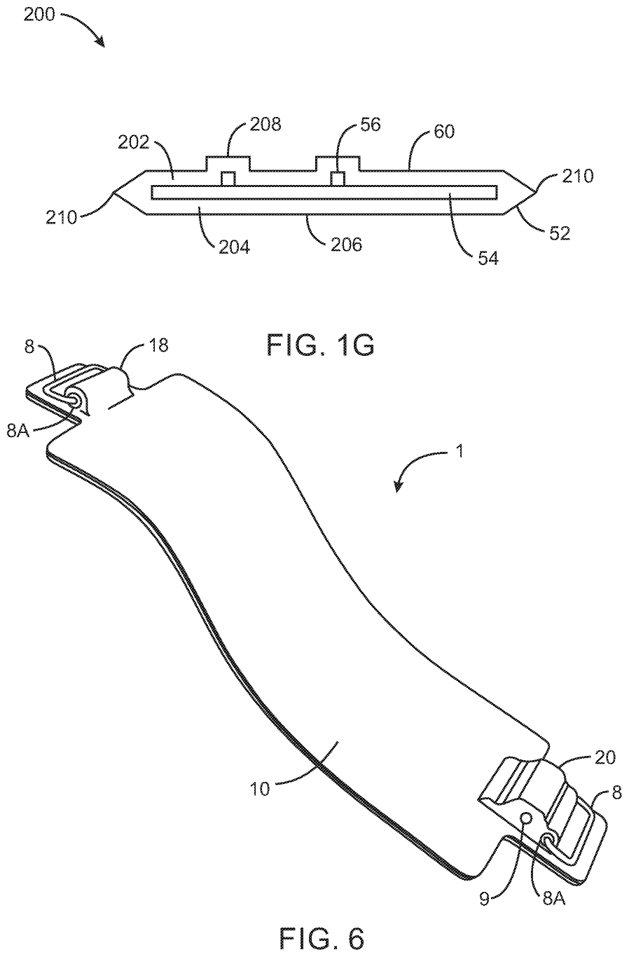

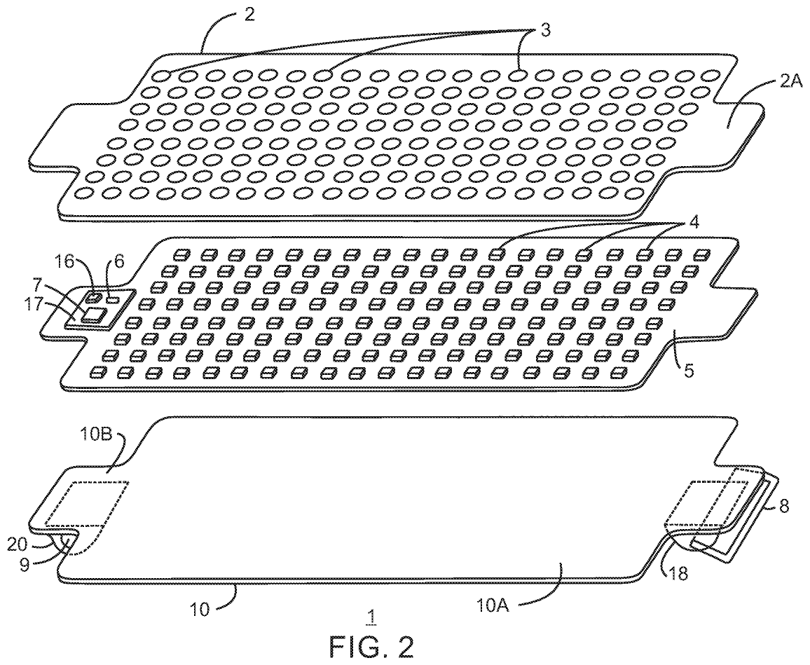

The same numbers are used throughout the disclosure and the figures to reference like components and features. Numbers in the 100 series refer to features originally found in FIG. 1; numbers in the 200 series refer to features originally found in FIG. 2; and so on.

### DETAILED DESCRIPTION

One or more specific embodiments of the present invention will be described below. In an effort to be concise, not all features of an actual implementation are described. It should be appreciated that in the development of any such actual implementation, as in any engineering or design project, numerous implementation-specific decisions must be made to achieve the developers' specific goals, such as compliance with constraints, which may vary. Moreover, it should be appreciated that such a development effort might be complex and time consuming, but would nevertheless be a routine undertaking of design, fabrication, and manufacture for those of ordinary skill in the art and having the benefit of this disclosure.

Some embodiments of the present techniques are a flexible pad and method. The pad including an internal layer having a flexible circuit board and having multiple light sources to emit light for light therapy treatment of a body part of a human or animal. A front exterior layer includes multiple raised portions to accommodate the multiple light sources. A back exterior layer in combination with the front exterior layer encases the internal layer, wherein the flexible pad is conformable to the body part. The flexible pad may be a flexible facemask pad or headgear (e.g., helmet) pad. The flexible pad may be shaped to fit a specific body part.

Certain examples are a flexible pad having: an internal layer comprising a flexible circuit board and having multiple light sources (e.g., at least 30 LEDs) to emit light having a wavelength in a range of 300 nanometers (nm) to 1100 nm for light therapy treatment of a human; a power input jack coupled to the flexible circuit board to receive power to the flexible pad, wherein the flexible pad accommodates a power supply that facilitates mobile application of the flexible pad in the light therapy treatment of the human; a front exterior layer comprising multiple raised portions to accommodate the multiple light sources, respectively, the front exterior layer to face a body part of the human in treatment; and a back exterior layer coupled to the front exterior layer, the back layer and the front layer in combination encasing the internal layer, wherein the flexible pad is conformable to the body part. Embodiments herein with the pad being “mobile” or “portable” means that an individual human can move the pad without the assistance of equipment, and that the pad is configured for mobile or portable use by a subject person or by a professional. Such gives unexpected results in ease-of-use including with respect to personal use. The front exterior layer and the back exterior layer may each be a flexible polymeric material such as silicone. The exterior surface of the front exterior layer may be bacterial resistant. The exterior surface of the back exterior layer may be generally flat. The front exterior layer and the back exterior layer may water resistant to provide for water-resistant operation of the flexible pad. The front exterior layer and the back exterior layer may be coupled via an adhesive in some examples.

The pad may include a pulse rate generator (PRG) to adjust operation of the flexible pad, and wherein to adjust operation includes to set operating modes of the flexible pad, the operating modes comprising multiple pulse rates, time duration, duty cycle, and energy settings. The PRG may be or include a device controller that operates a multiplicity of wavelengths, and also may be a master controlling one or more slave units. The pad may have a timer to set exposure time of the light emitted from the multiple light sources to the human. The PRG and the timer may be a single unit in some examples. Moreover, the PRG or the timer, or both, may be disposed along a power supply cord of the flexible pad. The flexible pad may include a power cord adapter to plug into the power supply to facilitate the mobile operation of the flexible pad. The pad may include an integrated circuit (IC) disposed on the flexible circuit board to secure power input jack (rigid) and/or an indicator light. The flexible pad may have a belt to hold the flexible pad adjacent the body part during the treatment. The belt may be coupled to the front exterior layer or the back exterior layer, or both. The flexible pad may have a belt coupler housing to facilitate receipt of the belt. The back exterior layer have the belt coupler housing. The belt coupler housing may have a pathway to receive the belt. The pad may have a ring disposed on the belt coupler housing to receive the belt. The belt coupler housing may be two housings, one disposed at each end portion of the flexible pad. The power input jack may disposed at least partially through the belt coupler housing.

Other aspects may include a method of operating a flexible pad in light therapy, the method including: positioning a surface of a front exterior layer of the flexible pad against a body part of a human, wherein the flexible pad conforms to the body part; coupling the flexible pad to a power supply; and turning on the flexible pad to emit light from a plurality of light sources on internal circuitry of the flexible pad, the light having a wavelength in a range of 300 nanometers (nm) to 1000 nm and directed through the front exterior layer to the body part to expose the light to the body part of the light therapy. The power supply may be a battery, a vehicle, a solar panel or other renewable energy source, or an electrical outlet on a wall in a building, and so on. The method may include securing the flexible pad adjacent the body part via a belt. The method may include setting a timer to specify a length of time of the exposure of the light to the body part. In some examples, the timer may have settings in a range of 15 minutes to 2 hours. The method may include adjusting operation of the flexible pad via a PRG.

Yet other aspects may include a method of manufacturing a flexible pad for light therapy of a human, the method including: obtaining a base polymeric material; obtaining a flexible circuit board comprising at least 3 light sources (or at least 10 light sources or at least 30 light sources) to emit light at a wavelength in a range of 300 nanometers (nm) to 1100 nm; and forming the base polymeric material around the flexible circuit board. The forming includes: forming a front exterior layer having raised portions to accommodate the light sources, wherein the light sources to emit the light through the front exterior layer to a body portion of the human for the light therapy; and forming a back exterior layer having a belt coupler housing to receive a belt, wherein the flexible pad is to conform to the body portion of the human for the light therapy. The forming may be molding (e.g., injection molding, liquid-silicone injection molding, etc.) the front exterior layer and molding the back exterior layer, and coupling (e.g., via an adhesive) the front exterior layer and the back exterior layer encasing the circuit board. The method may include curing the base polymeric material. The base polymeric material may be or include silicone, and may include an additive into the base polymeric material for bacteria resistance. The method may include providing the belt, and/or a power adapter to route electricity to the pad from a power supply comprising a vehicle, solar panel, battery, or electrical outlet. The method may include providing a timer to set an exposure time of the light to the body part, and/or providing a PRG to set an operating mode of the pad.

The present embodiments relate generally to light treatment techniques for pain therapy. Some examples include mobile techniques of applying light treatment therapy. Light-treatment devices may be employed for pain management, healing, elevating subject-tissue temperature, and other health and treatment considerations. Certain examples relate to mobile, flexible electronic-pad devices. Some pad devices include optional power-source interfaces facilitating the pad device to operate typically unrestricted as to location.

As indicated above and discussed below, a pad may include an interior or internal layer (or semi-internal layer) having a circuit board and multiple light sources to emit light for light therapy treatment of a body part of a human or animal. A front exterior layer includes multiple raised portions (e.g., translucent dimples) to accommodate the underlying multiple light sources. At least a portion of the front exterior layer including the raised portions may be clear or translucent to facilitate passage of the emitted light. A back exterior layer in combination with the front exterior layer may encase (or partially encase) the internal layer, wherein the pad is typically conformable to the body part. The pad may include additional layers. The pad may be shaped to fit a specific body part. For instance, the pad may be a facemask pad or head pad.

In examples, the pad having the circuitry and light sources may be semi-flexible or rigid. Also, the pad may be more stationary and less mobile. Examples of the pad may be implemented in-chair. In other words, the pad having the circuitry and light sources may be a component of a chair, bed, table, or other receptacle or holder in which a human patient may reside or be positioned to receive treatment. In some examples, the pad may be relatively stationary. In other examples, the pad may be moved or positioned over or under the patient. The pad may also be disposed inside a soft or hard cast, plastic or fabric injury brace.

An example is applying light treatment therapy from a pad having LEDs (e.g., 880 nm) affixed to a flexible circuit board (FCB). The FCB may be encased and insulated (e.g., water resistant) with flexible synthetic material. The pad may be portable and, therefore, the application of the light treatment therapy may be mobile as mentioned. Again, in certain examples, the flexible pad may be operated via multiple power-source interfaces facilitating the device to be used in the home and on the road, and in different places generally. To further facilitate home, office, emergency vehicle, and on-the-road implementation, the pad may be equipped with timer and/or pulse rate settings for ease-of-use by the end-user or subject. In sum, certain embodiments relate to a mobile, flexible electronic pad with power sources available facilitating the device to operate substantially unrestricted as to location. In examples, the generally lightweight flexible material and also operating ease of flexible-pad devices are features that promote the flexible pad as a mobile light-therapy device. This unique pad may provide for the user to decide when, where, and how much therapy to apply, as reasonable.

Conventionally, laser technology may address pain management with low-level wattage treatments for pain. However, cost may be high. Also, operation may be problematic and directed to a professional practitioner. The history of pain management has led technicians and manufacturers to collaborate and develop LED technology applications as an approach for pain treatment. Light sources such as light emitting diodes (LEDs) generate wavelengths of light (e.g., monochromatic light or polychromatic light) that may be generally beneficial in treatment. Yet, some LED treatment devices may be relatively large and cumbersome, and/or restricted to use at a physician's office. Moreover, some LED devices may lack mobility and flexible operation in treatment. In contrast, certain embodiments herein provide for mobile and varied treatment. Further, examples of LED-based devices herein may be in a shape or contour advantageous to treatment. Again, a very particular example of a present device utilizes 880 nm wavelength light, a wavelength that has shown to be effective for pain at the surface or shallow level, and for deep tissue and muscle penetration. Other wavelengths of light are applicable.

The application of light treatment therapy may employ multiple light sources (e.g., LEDs) affixed to a flexible circuit. In some examples, the wavelength of the light is in the range of 660 nm to 940 nm (e.g., is 880 nm). Moreover, in examples, the number of LEDs is 3, 10, 50, 80, 100, 130, 152, 200, 300, 500, 1000, or greater, or any number there between. The application of light treatment therapy by the circuitry configuration may be distributed generally evenly from the flexible pad in certain examples. On the other hand, the application of light via the circuitry configuration and the light sources may be more concentrated or more intense at a first portion of the flexible pad than at a second portion of the flexible pad. As indicated, the flexible pad may be mobile and capable to operate at home or in a vehicle, for example, or on its own battery powered supply and other power supplies.

FIG. 1A is an electronic pad 50 which may be rigid or flexible. In the illustrated example, the electronic pad is a flexible pad. The electronic pad has a body 52 with an internal circuitry 54 (e.g., including a flexible printed circuit board) having multiple light sources 56 (e.g., 10+ light sources 56). The light sources 56 may be LEDs and/or other types of light sources. The circuitry 54 may also include an integrated circuit (IC) 58 (e.g., disposed on the flexible printed circuit board) to facilitate various control and operational adjustment. A top or front exterior surface 60 may have raised portions (e.g., a dimpled surface) to accommodate the underlying light sources 56, respectively. The front exterior surface 60 may face the human body part during treatment. At least a portion of the front exterior surface 60 may be clear or translucent to allow passage of light from the light sources 56 through the surface 60 to the treatment subject. In alternate examples, the light sources 56 may protrude with no surface disposed between the light sources 56 and the patient. The rigid or flexible pad 50 may include a belt 64. In the illustrated example, the belt 64 is two-piece, with each piece affixed 63 at one end to the body 52. The belt may have coupling elements 64 (e.g., belt mechanism, rings, Velcro®, hook and loop, elastic, etc.) at the other ends to couple to those ends of the belt when applied to or wrapped around the subject body part.

The rigid or flexible pad 50 may include a power supply adapter 66 and cord to receive power from a power supply 68 (not shown). The power adapter 66 couples to the main body 52 and circuitry 54 to provide power to the circuitry 54 and the light sources 56. In the illustrated embodiment, the power adapter 66 plugs into a power input jack 70 affixed to the coupled to the circuitry 54 and accessible to the exterior of the main body 52. The pad 50 may also include a timer 72 and a PRG 74, both of which may be associated with the cord of the power adapter 66, as indicated by arrow 76. Examples of the timer and PRG are discussed below with respect to subsequent figures. A controller for other functions may be included.

FIG. 1B is an exemplary electronic pad 80 which is similar to the pad 50 of FIG. 1A, except that the belt 80 is a single piece loop and removable from the main body 52. In FIG. 1B, the main body 52 has belt coupler components 84 (e.g., housings, rings, etc.) to receive and guide (thread) the belt 82. Moreover, in this particular example, a power jack input 86 coupled to the circuitry 54 is disposed in or on one of the belt couple components 84. FIGS. 1C to 1F depict example electronic pads 90, 92, 94, and 96, respectively, and which share similar features with the electronic pads of the preceding figures. One difference is the geometry of the main body 52. The electronic pads 90, 92, 94, and 96 may be flexible pads 90, 92, 94, and 96.

FIG. 1G is a side cross-section view of an exemplary electronic pad 200 (e.g., rigid or flexible). The main body 52 includes an upper or front exterior layer 202 and a lower or back exterior layer 204. In the illustrated example, the front exterior layer 202 and the back exterior layer 204 in combination encase internal circuitry 54 (e.g., a flexible PCB). The back exterior layer has an exterior surface 206 that may be generally flat and/or smooth in certain examples. In contrast, the front exterior layer 202 has raised portions 208 to accommodate the light sources (e.g., LEDs) on the circuitry 54. The front layer 202 and the back layer 204 may be fabricated or formed (e.g., molded) together as a single molded form around the circuitry 54. On the other hand, the front exterior layer 202 and the back exterior layer 204 may be fabricated or formed (e.g., molded) as two respective components, and then coupled (e.g., via an adhesive) to each other. Of course, other fabrication configurations are applicable. The electronic pad 2 as a flexible pad may be shaped to fit a specific body part, and may be a facemask pad.

FIG. 1 is two perspective views of an exemplary electronic pad 1. The pad 1 may be a flexible electronic device for light treatment of humans or animals. If the pad 1 is flexible, the “flexible” with respect to the pad 1 is defined generally as conformable to a part or portion (e.g., back, neck, hip, shoulder, arm, elbow, leg, knee, crotch, face, head, etc.) of the human body for application of light. See, e.g., FIGS. 11A-12 and 14-20. The “flexible” pad 1 may contour or conform to the body part of the human or animal. The term “flexible” as used herein means to bend easily with individual human strength, and that the flexible pad has a very low Young's modulus, e.g., less than 100 megapascal (MPa). Moreover, again, while the pad 1 is depicted as generally rectangular in the illustrated embodiment, the pad 1 may be of other shapes and geometries, such as square, circular, oval, irregular, and so on. In one example, the flexible pad 1 is shaped as head gear (e.g., helmet or head flaps, etc.) to fit on a human head.

In certain embodiments, the pad 1 as illustrated in FIG. 1 has a length in the range of 5 centimeters (cm) to 100 cm or more, a width in the range 3 cm to 50 cm, a thickness (not including the belt housings at each end) in the range of 1 cm to 5 cm, and a weight (including the belt housings but not the belt or other accessories) in the range of 100 grams to 500 grams. Of course, embodiments of the pad 1 may have dimensions and weights outside of these ranges. In the particular illustrated example, the pad 1 has a length of 33 cm, width of 12.2 cm, and a thickness of 1.7 cm, and weighs 185 grams.

In addition to the circuitry including flexible circuitry, the material of the pad 1 may generally be rubber, silicone or silicone rubber, plastic (polymeric), metal, or cellulose, or any combination thereof. In certain embodiments, the pad layers (e.g., front and back layers) surrounding the electronics are rubber or flexible polymer such as silicone, polyethylene, polypropylene, or other plastic. In an embodiment, the pad exterior layers or covers are silicone.

Silicone may be polymerized siloxane elastomer, and substances whose molecules consist of chains made of alternating silicon and oxygen atoms. Silicones or polysiloxanes may be polymerized siloxanes. Indeed, silicones may be synthetic polymers with a silicon-oxygen backbone and may have organic groups attached to the silicon atoms by C—Si bonds, for example. The properties of a particular silicone may be tuned by changing the organic side groups attached to the silicon atoms. Silicone rubber compounds may consist of long-chain polysiloxanes and various fillers, such as pyrogenic silica, and which can be cured to form silicone elastomers. Silicone rubber may be an elastomer (rubber-like material) composed of polymer silicone containing silicon together with carbon, hydrogen, and oxygen.

In general, as discussed, the material of the pad 1 may be flexible (e.g., bendable) to contour to animal or human body parts, and to be rolled for easy storage, for instance, and the like. The exterior may be typically washable and water resistant, and in particular embodiments, the exterior may be bacterial resistant. Moreover, the pad 1 may be a facemask flexible pad or a headgear pad. In general, the pad may be shaped to fit a specific body part. For a headgear pad, the pad may be helmet or otherwise fit over the head or over a portion of the head. The headgear pad may have flaps (falling around the head) and/or slits, and the like, to facilitate placement of the headgear pad on the head.

In the illustrated example, the pad 1 has an exterior front layer 2 having an exterior front surface 2A that faces the patient during treatment. The front layer 2 and surface 2A may have raised portions 3 (e.g., dimples, square cubes, rectangular cubes, irregular cubes, pyramids, etc.) that coincide, respectively, with light sources 4 disposed underneath the front layer 2. At least a portion the front layer 2 and surface 2A (including the dimples 3) may be clear or translucent to allow for light to pass from the light sources 4 through the layer 2 and dimples 3.

In other examples, dimples 3 may not be employed but instead the light sources 4 extend or protrude through the surface 2A. Also, in some examples, the pad 1 may be rigid instead of flexible. Moreover, while depicted as stand-alone, the pad 1 may instead be an electronic pad 1 as a component of another device such as a treatment device.

FIG. 2 is an exploded view of an exemplary pad 1 which may be flexible or rigid.

Three layers 2, 5, and 10A are depicted. In the illustrated embodiment, the front layer 2 (the top or upper layer in the drawing) of the flexible pad 1 has a raised-dimpled top or front surface 2A with each dimple 3 accommodating or matching a light source 4 (e.g., a light emitting diode or LED) disposed underneath on internal circuitry of layer 5 (e.g., an interior layer, internal layer, partially-internal layer, etc.). The layer 5 may be an internal layer or a semi-internal layer. The layer 5 may flexible or rigid circuitry. In the illustrated example, the layer 5 is flexible circuitry.

In addition to the dimples 3 if employed, the exterior surface 2A of the front layer 2 may also be textured or otherwise have features for patient comfort and additional reasons. Indeed, in operation, the exterior surface 2A of the front layer 2 may face (be adjacent to or come in contact with) the human body portion being treated (in which light is applied) during treatment. As for the light sources 4 residing under the dimples 3, the light sources 4, in certain examples, are affixed to the interior layer 5 (e.g., circuitry) of the pad 1.

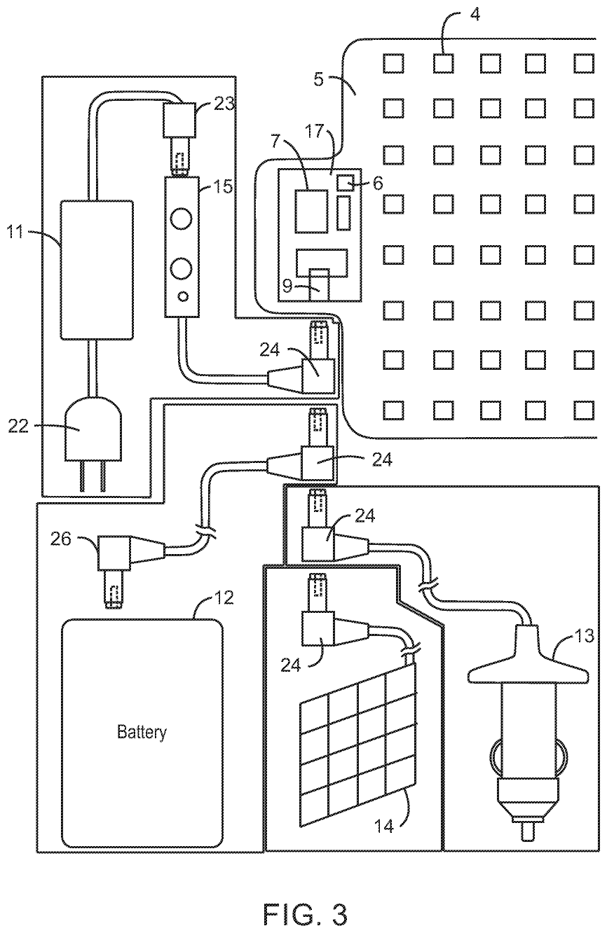

The interior layer(s) of the flexible pad 1 may be flexible circuitry 5 (a flexible circuit board) having the light sources 4, an indicator 6 (e.g., an indicator light or LED), an integrated circuit (IC) 7 (e.g., a more rigid circuit), a power input component 9 (e.g., jack, port, etc.), and so on. The IC 7 may provide for controls and also provide a more stable base for the power input component 9 and/or the indicator 6, for example. The indicator 6 may indicate when the flexible pad 1 is receiving power and/or emitting light via the light sources 4, and the like. In general, the indicator 6 may be an indicator light that is activated or illuminated when the flexible pad 1 is in operation. In other examples, in lieu of a light or in addition to the light, the indicator 6 may include a speaker for sound indication or alarm. Other indicator 6 types may be accommodated. Moreover, as discussed below with respect to FIG. 3, the power input component 9 may facilitate receipt and supply of electricity or power to the flexible pad 1.

As for the underlying circuitry, the circuitry 5 may have or receive (and hold) at least 20 light sources (e.g., LEDs). The light sources 4 may be disposed on the circuitry 5. The number of light sources 4 may range from 3 to 500, 10 to 500, or 30 to 500, for example. In the illustrated example, the flexible circuitry (5) accommodates 152 LEDs as the light sources 4. The number of light sources 4 on the flexible pad 1 may depend on the overall size (e.g., length and width) of the pad 1, the size and type of light source 4 employed, the desired distribution of the light emitted from the flexible pad 1 during treatment, the body part to be treated, and so forth. In some examples, the Hertz (Hz) associated with the LEDs may be in the range of 8 Hz to 20,000 Hz. If a flashing or strobe-like emission is implemented, the duty cycle (off/on) of the light emission may in the range of 1% to 99% time per cycle. Exemplary off/on times per cycle are 50%/50%, 40%/60%, 30%/70%, 20%/80%, 10%/90%. Again, these exemplary values are off/on light duration per cycle, and may be controlled by the circuitry 5, IC 7, PRG, timer, or other control device, or a coupled computing device, and so on.

In operation, the light sources 4 may emit light at various wavelengths including in exemplary ranges of the light emitted (in nanometers or nm) of 300 to 1200, 405 to 1100, 624 to 940, 660 to 940, 405 to 940, 660 to 880, 630 to 905, 450 to 660, or any nanometer values there between, and so on. Indeed, the numerical values of the wavelengths (nm) implemented may be individual values between the lower and upper values listed for these exemplary ranges. In a particular example, the light sources 4 are LEDs that emit 880 nm of monochromatic light. The wavelength (nm) values implemented may be based on economy, readily (commercially) available light-sources 4 (LEDs), wavelength values appropriate for treatment, and other factors. In particular examples, the flexible pad 1 may be labeled as an LED pad. Moreover, the wavelength (nm) of the light may be adjustable via controller or IC 7 (e.g., having a hardware processor and memory with stored code executable by the processor). The layer 5 or IC 7 may include a more rigid circuit board 17 for support and/or physical stability. The board 17 may be a part on the otherwise flexible circuit board 5. A network interface component 16 (e.g., Bluetooth® wireless) may be included. Further, the flexible pad 1 may include a data port or signal port to couple to a computer to receive information and data into memory. Such may be processed by the controller or IC 7, or by other circuitry on the flexible circuitry 5, and so on. The flexible pad 1 may include memory (e.g., nonvolatile memory, volatile memory, etc.) to store received data and information. The flexible pad 1 may include network interface circuitry or a network interface card to couple the pad with a network, such as a wired or wireless network. Various protocols may be accommodated such as Ethernet, Wi-Fi, Wireless Direct™ Bluetooth®, near field communication (NFC), and so on. Data and information may be downloaded from (or uploaded to) the network or internet. The pad 1 may be a smart device.

The back layer 10A may have a generally flat exterior surface 10. The surface 10 may be the backside of the flexible pad 1. In certain embodiments, the back layer 10A may be glued, stitched, sewn, fastened, or otherwise coupled to the top layer to enclose or encase (or partially encase) the flexible circuitry 5. The inner surface 10B of the back layer 10A may face the circuitry 5. As mentioned, the back layer 10A (and the front layer 2) may be rubber, flexible polymer, rigid polymer, metal, cellulose, or any combinations thereof. If polymer is used, the polymer may be silicone, polyethylene, polypropylene, copolymer, or other plastic. For a flexible pad, the material may be generally bendable to contour to animal or human body parts. In a particular example, the front and back layers are silicone, and at least a portion of the front layer 2 may be translucent silicone. However, other materials are applicable. The exterior surface 10 of the back layer 10A and the exterior surface 2A of the front layer 2 may be water resistant (and bacterial resistant in some embodiments). Again, the back layer 10A and the front layer 2 together may encase or enclose the internal layer 5 (circuitry 5) in certain embodiments.

A belt (not shown) may facilitate holding of the flexible pad 1 during operation against or around the human body part being treated. The belt may be one or two portions of belt, or more, depending on the configuration. Of course, options other than a belt may be employed to position the flexible pad 1 against the body portion or part being treated. In some examples, no belt or similar component is employed, but the pad 1 is merely placed against the body portion or part being treated. In other examples, a belt (or strap, rope, adhesive, flexible tube, elastic strap or band, etc.) may be associated with or part of the flexible pad 1, and affixed to the flexible pad 1, such as to the back layer 10A or front layer 2, or both. Two ends of the belt (the belt either as a loop or as two pieces) may couple or secure to each other such as with Velcro® surfaces on the belt, through a belt clamp or belt mechanism, tied, adhered, etc.

When the pad 1 is applied in treatment, the belt may at least partially secure the pad 1 to the human body (hold the pad adjacent the human body) during treatment. Indeed, as discussed, the pad 1 may contour to, or wrap around or partially around, a body part. If the pad 1 has a belt or if a belt is employed, the belt may facilitate holding of the pad 1 in place during the treatment. The material of the belt may be leather, fabric, plastic, silicone, cloth, adhesive, gauze, cast, brace material, and so on. The belt may be removable in embodiments. For instance, the front layer and/or back layer of the flexible pad 1 may include a belt coupler (e.g., fastener, clasp, clip, housings, ring, connector, Velcro® surface, etc.) that receives and engages the belt, and that disengages and releases the belt from the pad 1 when desired. In certain examples, a belt coupler is disposed on each end of the pad 1. The two or more belt couplers may each include a housing (e.g., having a hole, slot, or pathway) to receive the belt or to receive a ring that receives the belt. Indeed, belt coupler housings (belt loop housings) may be housings, one on each end of the pad, configured to receive a belt (loop) there through. The belt coupler housings may instead or further include a belt support such as a ring 8 (depicted in FIG. 2 on the right side only for clarity) or other support or guide to facilitate receipt, threading, and/or securing of the belt (not shown) to the respective housing and pad 1. Again, each end of the belt (loop) may have a belt end connector (which may be disengaged to release the belt) such a belt clamp or other belt mechanism, or Velcro® surfaces on the belt ends, or tied, etc. In a particular example, the housings 18 and 20 at each end of the pad 1, respectively, are two enclosed housings for utilization of a belt (e.g., Velcro® belt) that can be fitted when applying the pad 1 device to body parts. In the illustrated embodiment, one of the two belt loop housings has or surrounds a power input component 9 (e.g., power jack or port) for the selected power supply source. The power input component may be coupled or disposed on the internal layer 5.

In the illustrated example of FIG. 2, a belt coupler housing 18 is affixed or disposed to one end of the back layer 10A, and another belt coupler housing 20 is affixed or disposed to the other end of the back layer 10A. In some examples, the belt coupler housings 18 and 20 are molded as part of the back layer 10A (or front layer 2), e.g., in the molding of the back layer 10A (or front layer 2). The housings 18 and 20 may have receptacle (not shown) to receive or engage a clamp or ring 8, and with the belt threading the ring 8. The receptacle in or of the belt coupler housings 18 and 20 may be a hole, cylinder, tube, slot, etc. to receive the ring 8 that receives the belt. Lastly, one belt coupler housing 20 may be larger such that the housing 20 can accommodate multiple functions such as receiving the ring 8, and also encompassing the power input jack and the rigid circuitry (e.g., IC 7 and circuit board 17) in certain examples.

FIG. 3 is an exemplary flexible pad 1 with various power-supply options 11, 12, 13, and 14, and an optional operational control device or unit 15. The device 15 may have an on/off button or switch, a timer, a pulse rate generator (PRG), a controller, and so forth. The pad 1 may be configured for one or more of the depicted power-supply options 11, 12, 13, and 14, and other power-supply options. The power jack 9 and IC 7 may be disposed on and/or part of the internal circuitry 5. In examples, the jack 9 may be coupled to the IC 7. As mentioned, an indicator 6 (e.g., indicator light) notifying of the use or non-use of the unit may be couple to IC 7, or located at or near the end of the circuitry adjacent IC 7, for example. in certain embodiments, the indicator 6 can be seen or otherwise detected through top-layer material (e.g., silicone) in response to the pad 1 being in operation or use.

The input jack 9 and provided power supply cord/adapter unit may accommodate (plug into) various power supplies such as for home outlet 11, on its own battery pack supply 12, vehicle 13, solar panel 14, and so on. The power cord and adapter may be affixed to or plug into the flexible pad 1, and plug into a power supply (e.g., electrical outlet) at the home (or hospital, emergency vehicle, office, or business) via adapter 11, jack 23, and plug 22, or a battery pack supply 12 via adapter 26, an electrical supply adapter 13 for a vehicle, or coupled to solar panel 14, and the like. Other types of power cords/adapters and power supplied may be employed. In the illustrated example, the power cords have a 90-degree angled plug input 24 plug into the power input jack 9 of the pad 1. In some examples, the power cord integrates a device unit 15 (e.g., a timer, a detachable timer-PRG unit, etc.) for the user to set the time, operating mode, pulse width, and the like. In one example, the device unit 15 may provide for various modes, e.g., Pulse Mode, S (slow 73 Hz), M (medium 1,174 Hz) and F (fast 4,698 Hz)], etc. The Hz may range from 8 Hz to 30,000 Hz or greater. If the unit 15 incorporates a timer, the user may set the exposure time, such as in seconds, minutes, or hours (e.g., 15, 30, 45, 60, etc. minutes). The PRG and timer may be a single unit or separate units. Moreover, the unit 15 may be onboard of the circuitry 5 or IC 7. In some examples, the unit 15 may control the duty cycle discussed.

The unit 15 as a PRG may provide for the user to set pulse rates for different thresholds and to be specific to origin of pain, i.e., potential pain caused with skin, joints, muscles, bones, ligaments, nerves or inflammation. In some cases, the PRG 15 may include a timer, and the amount of exposure time may be differentiated, wherein shorter time with more repetition, another protocol might call for longer treatment time with less repetition. For a High-Low setting in particular example, the use on elderly or children or on dermal issues may be more appropriate. The high and low setting may be related to current amperage adjustments by the unit 15. The current to the light sources 4 (e.g., LEDs) may fall in ranges of 10 milliamp (mA) to 70 mA, 10 mA to 40 mA, 10 mA to 35 mA, and the like. A duty cycle setting may provide for long and short energy pulse output. The pad 1 may include thermistors (not shown) in-line on the power cord or unit 15 and/or on-board of the circuitry 5. The number of thermistors may be in the range of 5 thermistors to 300 thermistors, or other ranges. In one example, the flexible pad 1 has six rows of eight thermistors for a total of 48 thermistors. The thermistors may be controlled via the unit 15, and/or provide sensed feedback to the unit 15. The unit 15 may implement duty cycles ranging from 90% on/10% off to 10% on/90% off.

A thermistor is a temperature-sensing element composed of sintered semiconductor material which exhibits a large change in resistance proportional to a small change in temperature. Thermistors usually have negative temperature coefficients which means the resistance of the thermistor decreases as the temperature increases. Thermistors are made using a mixture of metals and metal oxide materials. Once mixed, the materials are formed and fired into the required shape. The thermistors can then be used “as-is” as disk-style thermistors. or further shaped and assembled with lead wires and coatings to form bead-style thermistors.

A thermistor is a type of resistor whose resistance is dependent on temperature, more so than in standard resistors. The word is a portmanteau of thermal and resistor. Thermistors are widely used as inrush current limiter, temperature sensors (NTC type typically), self-resetting overcurrent protectors, and self-regulating heating elements. Thermistors differ from resistance temperature detectors (RTDs) in that the material used in a thermistor is generally a ceramic or polymer, while RTDs use pure metals. The temperature response is also different; RTDs are useful over larger temperature ranges, while thermistors typically achieve a greater precision within a limited temperature range, typically −90° C. to 130° C.

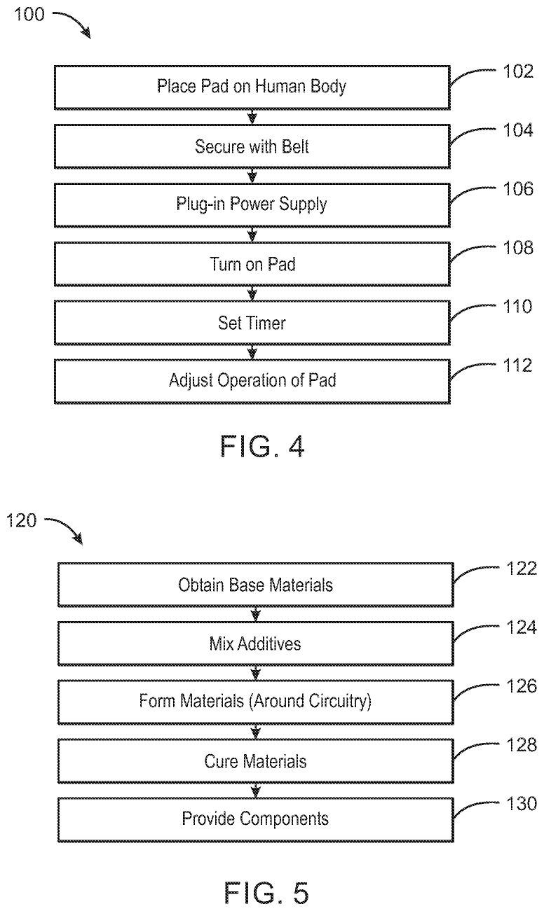

FIG. 4 is an exemplary method 100 of operating a pad for light therapy. While the discussion of FIG. 4 may focus on the pad as a flexible pad, a rigid or semi-rigid pad while likely not as conformable to some body parts may be similarly employed in general. The pad includes multiple light sources (e.g., 10, 50, 100, 1000, or more LEDs) that emit light toward the human or animal for the light therapy or treatment, such as to reduce pain. The light may increase temperature of the human or animal tissue. The user of the flexible pad employing the method 100 may be the human subject receiving the light therapy treatment (e.g., a typical person in the home or on the road using the flexible pad on themselves). The user may be a person other than the subject such as a doctor, nurse, professional, friend or family member, etc. employing the pad on the subject. The method 100 includes placing (block 102) the flexible pad adjacent or on a human (or animal) body, and in particular, on a portion or part of the body. Optionally, the flexible pad may be secured (block 108) via a belt to the body (e.g., the flexible pad wrapped around or partially around the body part). The user may plug (block 106) the flexible pad into a power supply. As discussed, a variety of power supplies may be utilized, and which may facilitate mobile use and application of the flexible pad. The power supply may be a battery or battery pack, a vehicle, a solar panel, an electrical outlet on a wall in a building, and so forth. The flexible pad may be turned on (e.g., via a switch) to admit power to the internal circuitry and lights for the pad to become operational and emit light. In some examples, simply plugging the flexible pad into the power supply may turn on the flexible pad.

As alluded, the method 100 in particular may include operating a flexible pad in light therapy, the method including: positioning (block 102) a surface of a front exterior layer of the flexible pad against a body part of a human, wherein the flexible pad conforms to the body part; coupling (block 106) the flexible pad to a power supply; and turning on (block 108) the flexible pad to emit light from a plurality of light sources on internal circuitry of the flexible pad, the light having a wavelength in a range of 300 nanometers (nm) to 1100 nm and directed through the front exterior layer to the body part to expose the light to the body part of the light therapy. The method may include securing (block 104) the flexible pad adjacent the body part via a belt.

Further, the method may include setting a timer to specify a length of time of the exposure of the light to the body part. In examples, the timer may have settings in a range of 5 minutes to 2 hours, or greater, e.g., 15 minutes, 30 minutes, 45 minutes, 1 hour, 90 minutes, and so on. Moreover, the method may include adjusting operation of the flexible pad via a pulse rate generator (PRG) if the pad has a PRG. Adjusting operation via the PRG may involve or include time, Hertz (Hz), high or low current or energy settings, duty cycle settings, and so on.

FIG. 5 is an exemplary method 120 of manufacturing a flexible pad for light therapy of a human or animal, the method including obtaining (block 122) a base material (e.g., a base polymeric material). The method 120 may also typically involve obtaining circuitry such as flexible circuit board. In certain embodiments, the method includes obtaining a flexible circuit board having at least 10, 50, 100, 1000, or more light sources (e.g., LEDs) to emit light at a wavelength in a range of 300 nanometers (nm) to 1100 nm. In a particular example, the flexible circuit board has or is configured to receive 152 LEDs. The method 120 may include incorporating or mixing (block 124) an additive or additives with the base polymeric material, to provide, for example, bacterial resistance and/or other properties.

The method 120 includes forming (block 126) the base polymeric material (e.g., silicone) around the flexible circuit board. Further, the method 120 may include curing (block 128) the base polymeric material, such as via treatment of an additive or catalyst, and/or heat, and the like. The forming may comprise injection molding (e.g., liquid-silicone injection molding) or other types of molding. The forming (block 126) of the base polymeric material (e.g., silicone) around the flexible circuit board may include: forming a front exterior layer have raised portions to accommodate the light sources, wherein the light sources to emit the light through the front exterior layer to a body portion of the human for the light therapy; and forming a back exterior layer, wherein the flexible pad is to conform to the body portion of the human for the light therapy. In certain embodiments, the back exterior layer is formed to have a belt coupler housing to receive a belt, for example. Moreover, the forming may include molding the front exterior layer and molding the back exterior layer, and coupling (e.g., via an adhesive) the front exterior layer and the back exterior layer encasing the flexible circuit board. Various equipment and unit operations may be employed to perform the forming and molding actions.

The flexible pad may be formed generally rectangular in shape, other geometries or shapes (e.g., circular, oval, square, irregular, etc.). In certain embodiments, the flexible pad has a length in a range of 5 centimeters (cm) to 100 cm, and a width in a range of 3 cm to 50 cm. The base thickness (not including the height of the belt coupler housings, for example) of the flexible pad may be in a range of 5 mm to 5 cm in some embodiments. In examples, the flexible pad weighs less than 500 grams. Indeed, the pad may be designed and fabricated as relatively light weight to facilitate the portable and mobile applicability and operation of the pad device.

Additionally, the method 120 may include providing (block 130) components such as the belt, a timer to set an exposure time of the light to the body part, a pulse rate generator to set an operating mode of the flexible pad, a power adapter including cord to route electricity to the flexible pad from a power supply. In some examples, the timer and PRG may be provide in the same housing. Moreover, in particular examples, the time and/or PRG may be provided as component(s) along the power cord. Further, the power adapter provided may be configured for receiving power from a vehicle, solar panel, a battery or battery pack (which may also be provided), an electrical outlet in a building, and so on.

The forming and molding may be, for example, via injection molding of liquid silicone rubber (LSR) or other polymeric materials. The molding actions may be implement with a variety of equipment and unit operations. For instance, such equipment may include an injection device pressurizing the liquid silicone to aid in the injection of the material into the pumping section of a molding machine. The pressure and injection in some examples may be adjusted at the operator's discretion. The equipment may include metering units to pump primary liquid materials, such as the catalyst and the base forming silicone, to facilitate that the two materials maintain a substantially constant ratio while being simultaneously released. Supply drums, also called plungers, may serve as the containers for mixing materials. Both the supply drums and a container of pigment, for instance, may connect to a main pumping system. Mixers, such as a static or dynamic mixer, may combine materials after they exit the metering units. Once combined, pressure may drive the mixture into a designated mold. A nozzle may facilitate deposition of the mixture into the mold. If a nozzle is used, the nozzle may feature an automatic shut-off valve to help prevent or reduce leaking and overfilling the mold. The molding fabrication system may also generally include a mold claim, and other components.

FIG. 6 is an example of the flexible pad 1 (see FIGS. 1-3 for reference numerals). In this perspective view of FIG. 6, the backside (surface 10) of the flexible pad 1 is on the top. As can be seen, belt coupler housings 18 and 20 are disposed, respectively, on each end (e.g., molded as part of the back layer 10A). Each belt coupler housing 18 and 20 may also have a ring 8, as depicted, to receive a belt (e.g., a belt loop). The ring 8 may be metal, fabric, cloth, plastic, or other material. The housings may have a receptacle 8A (e.g., hole, cylinder, depressed area, pathway, etc.) to receive or secure the ring 8. Moreover, as also depicted, the belt coupler housing 20 on the right side is larger (relative to the belt coupler housing 18 on the left side) so to incorporate the power input jack 9 (and also to incorporate an optional indicator light 6 not shown in this particular example). Moreover, in this illustrated embodiment, the back and front exterior layers of the pad 1 are constructed of silicone.

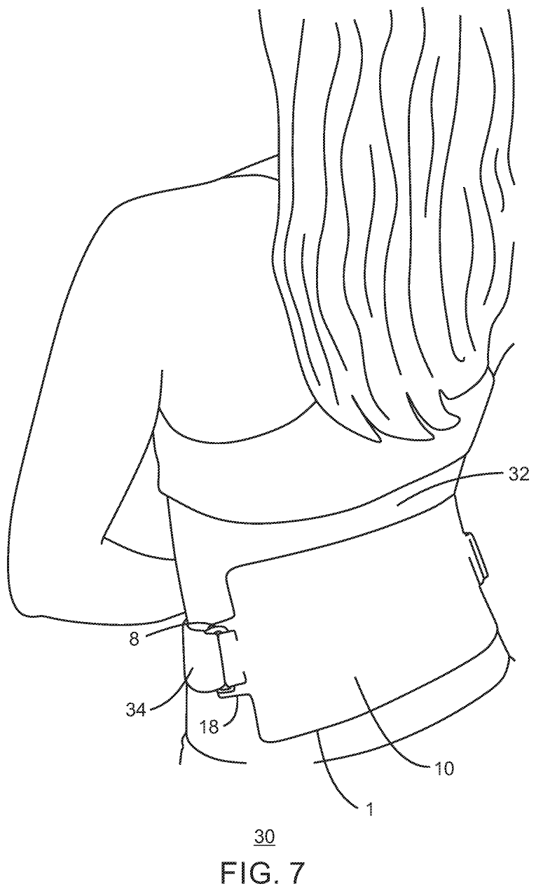

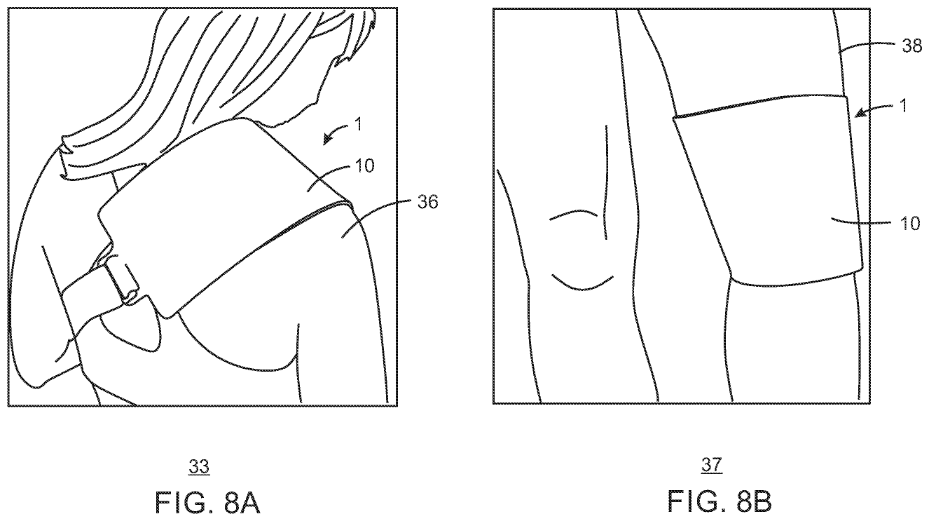

FIGS. 7, 8A, and 8B depict respective applications of the flexible pad 1 to different portions or body parts of the human body. In these examples, a belt 34 is employed. The belt 34 may be secured via a ring 8 to belt coupler housings (e.g., 18) on the flexible pad 1. The belt 34 may be fabric and have, for example, Velcro® or a belt buckle to couple loose ends of the belt 34 to form a belt 34 loop. In FIG. 7, the pad 1 is applied to the back 32 of a person. In FIG. 8A, the pad 1 is applied to a shoulder 36 of a person. In FIG. 8B, the pad is applied to a knee 38 of a person.

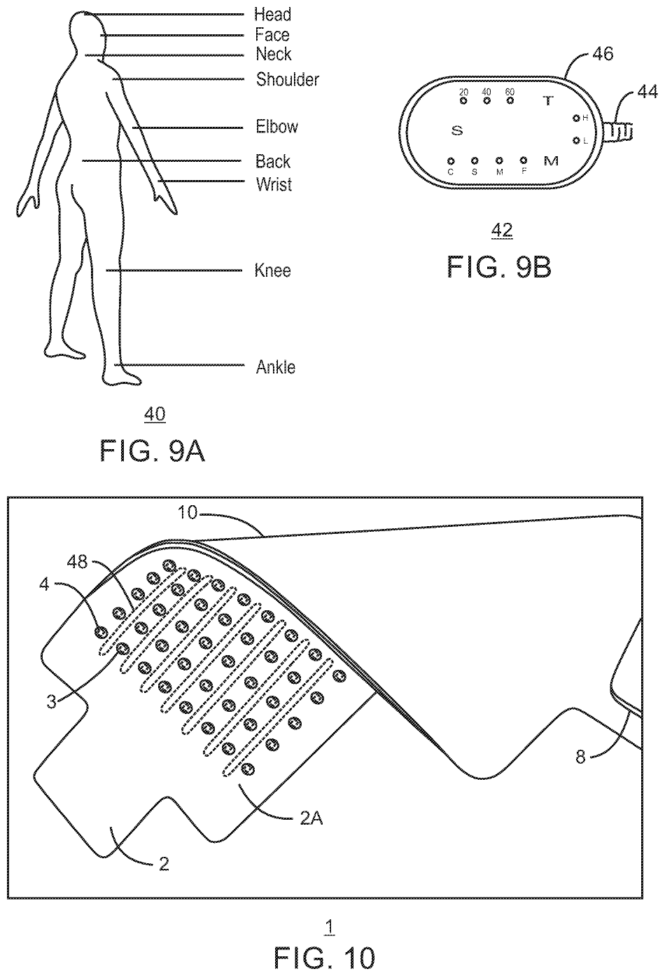

FIG. 9A depicts a human subject and example body parts or portions that may be treated with the flexible pad 1. Such parts may include head, face, neck, shoulder, elbow, back, wrist, knee, ankle, and other body parts. Light therapy may relieve pain, reduce inflammation, enhance cell function, accelerate healing or recovery, and so forth. The light therapy may be implemented in conjunction with nitric oxide activation. Supplements taken by or applied to the human subject may generate or activate nitric oxide in the body. The increased presence of nitric oxide may work in conjunction with the light to advance pain reduction and healing via the light therapy. The activating of nitric oxide at the cellular level may increase the body's ability to reach homeostasis. This action may encourage cells back to their original healthy state. Nitric oxide activation or nitric oxide supplementation may assist in healing, improve blood circulation, increase oxygen delivery, and so on.

FIG. 9B is an exemplary control unit 42 and may be analogous the timer 72 and/or (PRG) 74 of FIGS. 1A-1F, the operational control unit 15 of FIG. 3, and other control units The unit 42 may have an input jack or adapter 44 to couple to a pad 1 or to a power cord of the pad 1. In other examples, the control unit 42 may be disposed directly on the pad 1 and internally coupled to the flexible pad 1. The control unit 42 may have a housing 46 surrounding one or more circuits. Depending on the version of the control unit 42 implemented, the control unit 42 may have a start S capability, as well as a high H and low L settings of the intensity of the emitted light. The unit 42 may provide for duty cycle control with adjustment of the duty cycle of the light emission. The control unit 42 may be a timer or include a timer (labeled as “T”) in which the user may be able to set the time of light emission of the flexible pad 1. The illustrated example shows timer settings of 20, 40, and 60 minutes. Other time settings may be incorporated. In some examples, the unit 42 may incorporate a PRG with pulse mode settings of constant C, slow S (e.g., 72 hertz or Hz), medium M (e.g., 1,174 Hz), and fast F (4,698 Hz). Other PRG settings, and other settings generally and control capabilities, are applicable.

FIG. 10 is an exemplary pad 1 having a backside surface 10. The front surface 2A of the front exterior layer 2 has dimples or raised portions 3 to accommodate (provide a cavity or space for) the underlying respective light sources 4 (e.g., LEDs) disposed on an interior layer. At least a portion of the layer 2 may be clear or translucent. An internal stitching 46 may facilitate coupling of the exterior layer 2 to an internal layer, or included for aesthetics.

FIG. 11A is a front view of an exemplary flexible facemask pad 140. FIG. 11B is a back view of the facemask flexible pad 140. The facemask pad 140 has raised portions 3 on the front surface that cover respectively light sources on an internal layer. In the illustrated embodiment, the facemask pad 140 has four neck flaps 142 with a slit 144 between two neck flaps 142 on each side. The facemask pad 140 may have an opening 146 for the mouth and/or nose. The area around the opening 146 may be more flexible or adjustable to better accommodate the nose, for instance. In the illustrated example, slits 148 and 150 facilitate placement or folding in of the facemask pad 140 onto the human face. Moreover, in this example, coupler housings or rings 8 on the back near the perimeter may receive a belt to hold the facemask pad 140 on the human face. In other words, the belt may thread through the rings and around the back of the human head, for example.

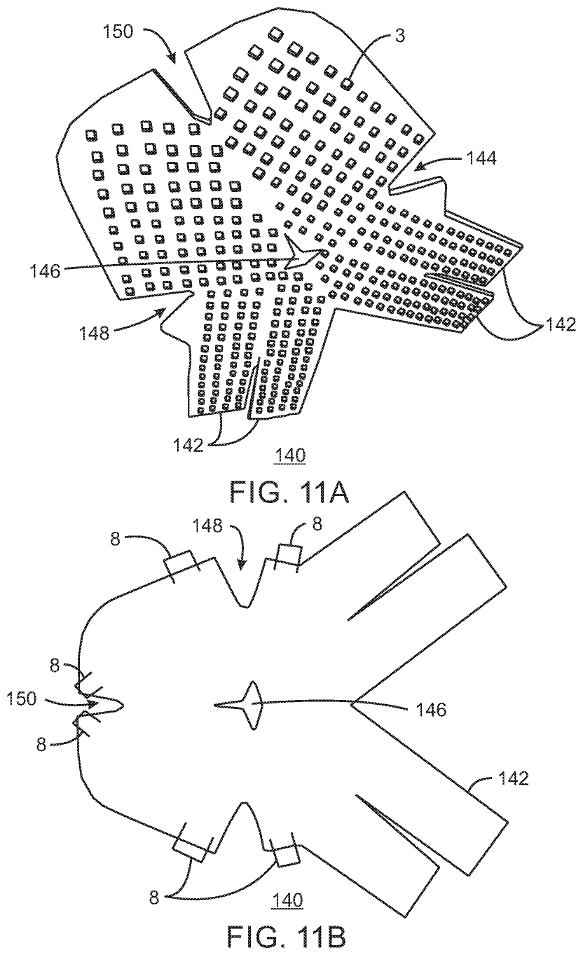

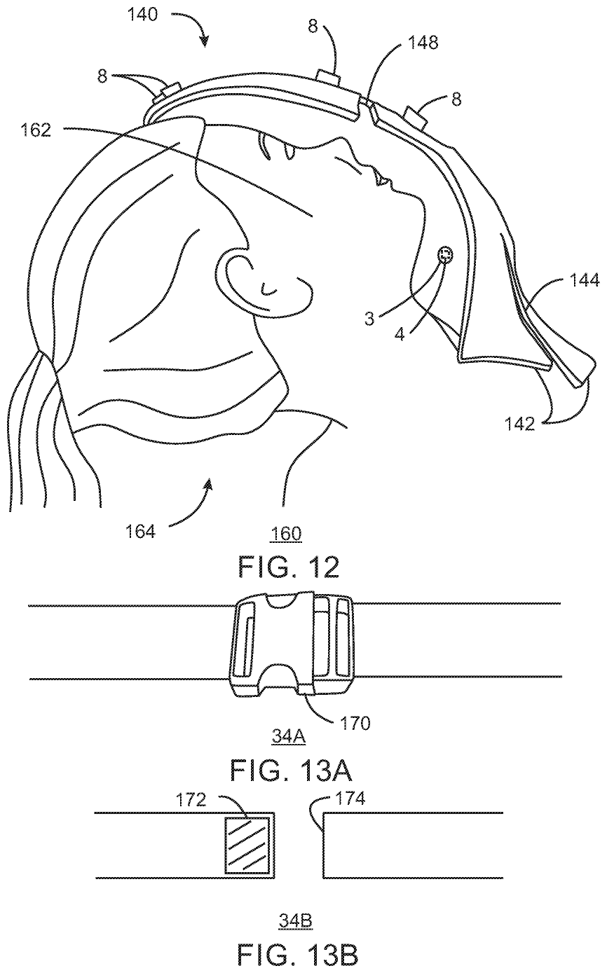

FIG. 12 is a perspective view of an application 160 of the flexible facemask pad 140 of FIG. 11 to a face 162 of a human 164. The flexible facemask pad 140 (facial mask) is an embodiment of the flexible light pad 1 having the layers and components discussed above with respect to the preceding figures. Indeed, the flexible facemask pad 140 has multiple raised portions 3 on the front surface and multiple corresponding lights sources 4 (e.g., LEDs) such as on an internal layer. The facemask flexible pad 140 may be a generally flat flexible material with a specific shape(s) and design(s) that included panels and openings in order to contour to multiple facial shapes and sizes. The apparatus is meant to be placed on face 162 and secured into place with hook and loop trough rings (e.g., rings 8) attached or embedded into the mask 140. In some instances, elastic bands may be used. These securing systems facilitate the mask 140 to be adjusted to fit contours of different facial sizes and shapes, facilitating the mask 140 to tighten and secure in place promoting light distribution on most or all areas of the face 162. As indicated above, the mask 140 may have a nose and/or mouth opening with two single flaps resting on each side of nose, for example. An opening at the mouth to may promote easy breathing. The mask 140 objective may be to reduce pain for several conditions such as pain associated with facial surgery with respect to an auto accident, sports injury or fibromyalgia, and so on, as well as post-surgical in cases of cosmetic, dental, sinus medical procedures, etc.

FIG. 13A is a belt 34A having a buckle 170 to removably couple or secure ends of the belt to each other. FIG. 13B is a belt 34B having Velcro 172 on one side of an end of the belt 34B and Velcro 174 on the other side of the other end of the belt 34B, such that the belt 34B ends may be removably coupled to each other. The Velcro 172 may mate with the Velcro 174.

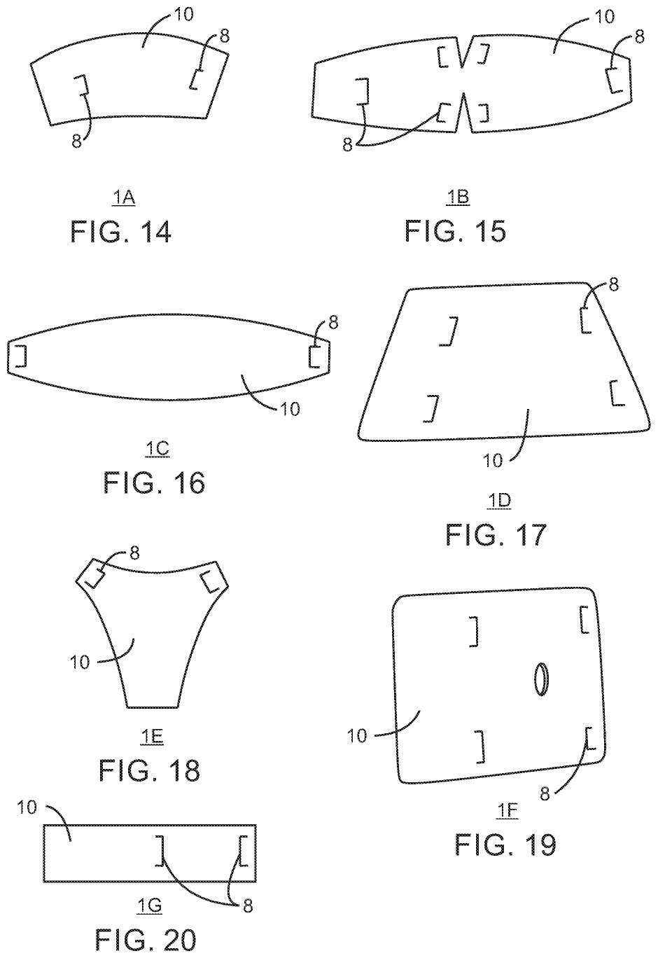

FIGS. 14-20 are back views of several different flexible pads 1A, 1B, 1C, 1D, 1E, 1F, and 1G to fit different specific human body parts, respectively. The electronic flexible pads may be custom for specific body parts. Again, the views are of the backside surface 10 of the pads. The pads have the light sources (not shown) and other features discussed above. If a belt or strap is employed, the pads may have coupler housing, receptacles, or rings 8, and the like to engage the belt. As for custom shape of the pad in the illustrated examples, the pad 1A may be for the ankle or neck, the pad 1B may for the knee, the pad 1C may be for the elbow or shoulder, the pad 1D may be for the foot, the pad 1E for the crotch, the pad 1F for the hand, and the pad 1G for the wrist or neck. A particular pad may be a skull cap pad configured to fit on and cover the skull or top of the head. The pad may be material as pizza wedge shapes strewn together with points of wedges at top. Rings and/or Velcro may be employed. The pad might be used for hair loss, Alzheimer's, headache, post-brain surgery, brain injury, Parkison's, and so on.

In summary, certain embodiments may be a flexible pad having: an internal layer comprising a flexible circuit board and having multiple light sources (e.g., at least 30 LEDs) to emit light having a wavelength in a range of 300 nanometers (nm) to 1100 nm for light therapy treatment of a human; a power input jack coupled to the flexible circuit board to receive power to the flexible pad, wherein the flexible pad accommodates a power supply that facilitates mobile application of the flexible pad in the light therapy treatment of the human; a front exterior layer comprising multiple raised portions to accommodate the multiple light sources, respectively, the front exterior layer to face a body part of the human in treatment; and a back exterior layer coupled to the front exterior layer, the back layer and the front layer in combination encasing the internal layer, wherein the pad is conformable to the body part.

The flexible pad may include a power cord adapter to plug into the power supply to facilitate the mobile operation of the flexible pad. The flexible pad may include a PRG to adjust operation of the flexible pad, such as to set operating modes of the flexible pad, the operating modes including, for example, time, Hz, duty cycle, high or low current or energy settings, and so on. Further, the pad may include a timer to set exposure time of the light emitted from the multiple light sources to the human. In some examples, the PRG and the timer are a single unit. Furthermore, in examples, the PRG or the timer, or both, are disposed along a power supply cord of the pad. The pad may include an IC disposed on the flexible circuit board to for at least the reason to provide a more stable base for the power input jack and/or indicator light.

The front exterior layer and the back exterior layer each comprise a flexible polymeric material. At least one of the front exterior layer or the back exterior layer may be or include silicone or silicone rubber. In some examples, an exterior surface of the front exterior layer (and/or back exterior layer) is bacterial resistant. Moreover, in examples, an exterior surface of the back exterior layer is generally flat. Furthermore, the front exterior layer and the back exterior layer may be water resistant to provide for water resistant operation of the flexible pad. The surfaces may an easy-cleaning surface and can be solvent-friendly for cleaning in certain examples. Also, the front exterior layer and the back exterior layer may be coupled to each other via an adhesive. Additionally, the flexible pad may include a belt to hold the flexible pad adjacent the body part during the treatment. The belt may be coupled to the front exterior layer or the back exterior layer, or both. The flexible pad may have a belt coupler housing to facilitate receipt of the belt. In some examples, the back exterior layer comprises the belt coupler housing. The belt coupler housing may have a pathway to receive the belt, and/or a ring disposed on the belt coupler housing to receive the belt. In certain embodiments, the belt coupler housing comprises two housings, one disposed at each end portion of the flexible pad. In an example, the power input jack is disposed at least partially through a belt coupler housings. Also, a light indicator to indicate when the flexible pad is in operation may be coupled to the internal circuitry and disposed in one of the belt coupler housings or elsewhere on the pad.

Some embodiments may be implemented in one or a combination of hardware, firmware, and software. Some embodiments may also be implemented as instructions stored on a machine-readable medium, which may be read and executed by a computing platform to perform the operations described herein. An embodiment is an implementation or example. Reference in the specification to “an embodiment”, “one embodiment”, “some embodiments”, “various embodiments,” or “other embodiments” means that a particular feature, structure, or characteristic described in connection with the embodiments is included in at least some embodiments, but not necessarily all embodiments, of the present techniques. The various appearances of “an embodiment,” “one embodiment,” or “some embodiments” are not necessarily all referring to the same embodiments. Elements or aspects from an embodiment can be combined with elements or aspects of another embodiment. Not all components, features, structures, characteristics, etc. described and illustrated herein need be included in a particular embodiment or embodiments. If the specification states a component, feature, structure, or characteristic “may”, “might”, “can” or “could” be included, for example, that particular component, feature, structure, or characteristic is not required to be included. If the specification or claim refers to “a” or “an” element, that does not mean there is only one of the element. Lastly, those skilled in the art having the benefit of this disclosure will appreciate that many other variations from the foregoing description and drawings may be made within the scope of the present techniques. Accordingly, it is the following claims including any amendments thereto that define the scope of the present techniques.

What is claimed is:

1. A flexible pad comprising:
an internal layer comprising a flexible circuit board and having multiple light sources to emit light having a wavelength in a range of 300 nanometers (nm) to 1100 nm for light therapy treatment of a human;
a power input jack coupled to the flexible circuit board to receive power to the flexible pad, wherein the flexible pad accommodates a power supply that facilitates mobile application of the flexible pad in the light therapy treatment of the human;
a front exterior layer comprising multiple raised portions to accommodate the multiple light sources, respectively, the front exterior layer to face a body part of the human in treatment; and
a back exterior layer, wherein the flexible pad is conformable to the body part.

2. The flexible pad of claim 1, comprising a timer to set exposure time of the light emitted from the multiple light sources to the human, wherein the front exterior layer and the back exterior layer in combination encase the internal layer, and wherein the front exterior layer and the back exterior layer each comprise a flexible polymeric material.

3. The flexible pad of claim 1, comprising a power cord adapter to couple to the power supply to facilitate the mobile operation of the flexible pad, wherein the multiple light sources comprise at least 10 light emitting diodes (LEDs), wherein the back exterior layer is coupled to the front exterior layer, and wherein a timer is disposed along a power supply cord of the flexible pad.

4. The flexible pad of claim 1, comprising an integrated circuit (IC) disposed on the flexible circuit board that secures a power input component or an indicator that indicates when the flexible pad is in operation, or both, wherein the front exterior layer and the back exterior layer are coupled via an adhesive.

5. The flexible pad of claim 1, comprising a belt to hold the flexible pad adjacent the body part during the treatment, wherein the flexible pad is shaped to fit the body part.

6. The flexible pad of claim 1, comprising:
a belt to hold the flexible pad adjacent the body part during the treatment; and
a belt coupler housing to facilitate receipt of the belt.

7. The flexible pad of claim 6, wherein the belt coupler housing comprises a pathway to receive the belt.

8. The flexible pad of claim 6, comprising a ring disposed on the belt coupler housing to receive the belt.

9. The flexible pad of claim 6, wherein the belt coupler housing comprises two housings, one disposed at each end portion of the flexible pad.

10. The flexible pad of claim 6, wherein a power input component is disposed at least partially through the belt coupler housing to facilitate physical stability of the power input component.

11. The flexible pad of claim 1, wherein the flexible pad is facemask pad or a headgear pad.

12. A method of operating an electronic pad in light therapy, the method comprising:
positioning a surface of a front exterior layer of the electronic pad against or adjacent a body part of a human;
coupling the electronic pad to a power supply; and
turning on the electronic pad to emit light from a plurality of light sources on internal circuitry of the electronic pad, the light having a wavelength in a range of 300 nanometers (nm) to 1000 nm and directed through the front exterior layer to the body part to expose the light to the body part of the light therapy.

13. The method of claim 12, comprising securing the electronic pad adjacent the body part via a belt, wherein the electronic pad comprises a flexible pad, and wherein the flexible pad conforms to the body part.

14. The method of claim 12, comprising setting a timer to specify time of exposure of the light to the body part, wherein the electronic pad is shaped to fit a specific body part.

15. The method of claim 12, wherein the power supply comprises a battery, a vehicle, a solar panel, or an electrical outlet on a wall in a building, or any combination thereof.

16. A method of manufacturing a flexible pad for light therapy of a human, the method comprising:
obtaining a base polymeric material;
obtaining a flexible circuit board comprising at least ten light sources to emit light at a wavelength in a range of 300 nanometers (nm) to 1100 nm; and
forming the base polymeric material around the flexible circuit board, comprising:
forming a front exterior layer have raised portions to accommodate the light sources, wherein the light sources to emit the light through the front exterior layer to a body portion of the human for the light therapy; and
forming a back exterior layer comprising a belt coupler housing to receive a belt, wherein the flexible pad is to conform to the body portion of the human for the light therapy.

17. The method of claim 16, comprising curing the base polymeric material, wherein the forming comprises injection molding, wherein the flexible pad is shaped to fit a body part.

18. The method of claim 16, wherein the forming comprises molding the front exterior layer and molding the back exterior layer, and coupling the front exterior layer and the back exterior layer encasing the flexible circuit board.

19. The method of claim 18, wherein the coupling comprises coupling, via an adhesive, the front exterior layer and the back exterior layer.

20. The method of claim 16, comprising providing the belt, providing a power adapter to route electricity to the flexible pad from a power supply, and providing a timer to set an exposure time of the light to the body part.
